(19) United States
(12) Patent Application Publication
(10) Pub. No.: US 2023/0373938 A1
(43) Pub. Date: Nov. 23, 2023

(54) BENZOTHIAZOLE MEROTERPENOID COMPOUND AND DERIVATIVE THEREOF, AND PREPARATION METHOD THEREFOR AND USE THEREOF

(71) Applicant: THIRD INSTITUTE OF OCEANOGRAPHY, MINISTRY OF NATURAL RESOURCES, Xiamen (CN)

(72) Inventors: Xianwen YANG, Xiamen (CN); Chunlan XIE, Xiamen (CN); Qingxiang YAN, Xiamen (CN); Zhenbiao ZOU, Xiamen (CN); Zhihui HE, Xiamen (CN)

(73) Assignee: THIRD INSTITUTE OF OCEANOGRAPHY, MINISTRY OF NATURAL RESOURCES, Xiamen (CN)

(21) Appl. No.: 18/027,379

(22) PCT Filed: Mar. 18, 2021
(86) PCT No.: PCT/CN2021/081434

§ 371 Date: Mar. 21, 2023

(30) Foreign Application Priority Data
Sep. 21, 2020 (CN) 202010995177.5

(51) Int. Cl. C07D 277/68; C12P 17/14; C12N 1/14; A61P 35/00

(52) U.S. Cl. CPC C07D 277/68; C12P 17/14; C12N 1/14; A61P 35/00

(57) ABSTRACT

A benzothiazole meroterpenoid compound derived from deep sea fungi and a derivative thereof, and a preparation method therefor and the use thereof in the preparation of an anti-tumor drug are provided. The benzothiazole meroterpenoid compound is a compound as represented by formulas I to III or a salt thereof. The compound is isolated from a fermentation product of deep-sea-derived Penicillium allii-sativi and inhibits the transcriptional activity of an anti-tumor target RXRalpha by means of binding thereto so as to achieve a remarkable anti-tumor effect. The compound can be used for preparing, researching and developing anti-cancer drugs, and has good application prospects.

### CROSS REFERENCE TO THE RELATED APPLICATIONS

This application is the national phase entry of International Application No. PCT/CN2021/081434, filed on Mar. 18, 2021, which is based upon and claims priority to Chinese Patent Application No. 202010995177.5, filed on Sep. 21, 2020, the entire contents of which are incorporated herein by reference.

### TECHNICAL FIELD

The present invention relates to the technical field of pharmaceutical compounds, and in particular, to a benzothiazole meroterpenoid compound and a derivative thereof, and a preparation method therefor and the use thereof.

### BACKGROUND

Tumor is one of the most serious diseases that affect human health. Nuclear receptors are a family of ligand-dependent regulatory transcription factors that are distributed in cytoplasm and nucleus of cells and play an important role in maintaining homeostasis. As an important member of the nuclear receptor family, retinol X receptor (RXR) is considered to be the most promising core member that plays an important role in regulating proliferation and apoptosis of various cancer cells such as lung cancer cells, breast cancer cells, liver cancer cells and prostate cancer cells. RXRalpha contains an N-terminal region, a DNA binding domain and a ligand binding domain (LBD). RXRalpha-LBD has a ligand binding pocket (LBP) for binding small molecules with ligands, and it is able to recognize specific hormonal and non-hormonal ligands. RXR ligands can regulate RXR-related signaling pathways by means of activation or antagonism, and both means can play a role in metabolic diseases or cancer to varying degrees. Therefore, the discovery of RXRalpha ligands is a hot research topic. In the previously reported RXRalpha-LBD ligands (such as all-trans retinoic acid, 9-cis sulfonic acid, targeted Leiding, CD3254, K8003, K8008), there are three basic regions: a hydrophobic group, a polar region and a central polyene bond linker structure.

Although many natural ligands of RXRalpha have been reported, their selectivity is poor and toxicity is high. Marine microorganisms can produce secondary metabolites with novel structures due to their existence in a special marine environment, and these secondary metabolites (such as alkaloids, terpenoids and flavonoids) from the sea can exhibit high anti-tumor activity. Therefore, the discovery of high-efficiency and low-toxicity RXRalpha small molecule regulators from natural products is an effective strategy for the development of anti-tumor drugs based on RXRalpha targets.

### SUMMARY

An objective of the present invention is to provide a benzothiazole meroterpenoid compound and a derivative thereof, and a preparation method therefor and the use thereof. The benzothiazole meroterpenoid compound and the derivative thereof are isolated from a fermentation product of Penicillium allii-sativi so as to achieve a remarkable anti-tumor activity. The compound can be used for preparing, researching and developing anti-cancer drugs.

Therefore, according to a first aspect, the present invention provides a benzothiazole meroterpenoid compound and a derivative thereof. The benzothiazole meroterpenoid compound is a compound as represented by formulas I to III or a salt thereof:

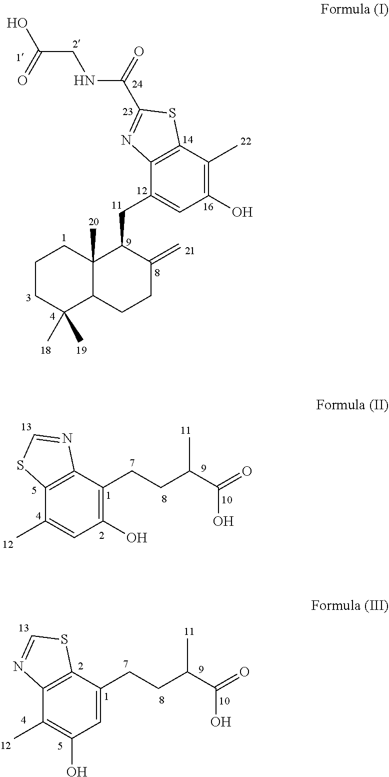

According to a second aspect, the present invention provides a preparation method for the benzothiazole meroterpenoid compound and the derivative thereof, including the following steps:
S1. fermenting Penicillium allii-sativi to obtain a fermentation product; where the Penicillium allii-sativi is deposited in Marine Culture Collection of China (MCCC), located at No. 178 Daxue Road, Siming District, Xiamen City, Fujian Province, 361005 with collection No. of MCCC 3A00580, and deposited on Dec. 31, 2014; and
S2. extracting the fermentation product obtained in step S1, and isolating and purifying a resulting extract to obtain the benzothiazole meroterpenoid compound and the derivative thereof.

Preferably, the step S2 includes:
S21. extracting the fermentation product obtained in the step S1, extracting the fermentation product with ethyl acetate, taking an organic extract for chromatography, and eluting with petroleum ether, dichloromethane and methanol respectively; and concentrating a dichloromethane layer to obtain a crude extract;
S22. isolating the crude extract obtained in the step S21 by normal phase silica gel column chromatography, and performing gradient elution by using a petroleum ether-ethyl acetate system to successively obtain eight crude fractions: Fr.1-Fr.8;
S23. isolating the crude fraction Fr.5 obtained in the step S22 by ODS column chromatography, and performing gradient elution by using a water-methanol system to successively obtain eleven crude fractions: Fr.5.1-Fr.5.11;
S24. isolating the crude fraction Fr.5.9 obtained in the step S23 by using a sephadex column and a semi-preparative liquid chromatographic column to obtain a compound as represented by formula I;
S25. isolating the crude fraction Fr.6 obtained in the step S22 by sephadex column chromatography to obtain three crude fractions: Fr.6.1-Fr.6.3; and
S26. isolating the crude fraction Fr.6.2 obtained in the step S25 by ODS column chromatography and semi-preparative liquid chromatography to obtain compounds as represented by formula II and formula III.

Further, an elution solvent for normal phase silica gel column chromatography used in the step S22 is a petroleum ether-ethyl acetate system at a ratio of 100:1, 50:1, 30:1, 10:1, 5:1, 3:1 and 1:0.

Further, in the step S23, a mobile phase used in the ODS column is methanol-water with gradient elution (40%→100%, 15×310 mm, 20 ml/min).

Further, in the step S24, a mobile phase used in the sephadex column is methanol, and a mobile phase used for the semi-preparative liquid chromatographic column is acetonitrile-water with gradient elution (ACN—H₂O, 60%→100%, 10×250 mm, 5 ml/min).

Further, in the step S25, a mobile phase used in the sephadex column is methanol.

Further, in the step S26, a mobile phase used in the ODS column is methanol-water with gradient elution (40%→100%, 15×310 mm, 20 ml/min); and a mobile phase used in the semi-preparative liquid chromatographic column is acetonitrile-water with gradient elution (ACN—H₂O, 40%→100%, 10×250 mm, 5 ml/min).

Preferably, in the step S1, fermentation conditions for the Penicillium allii-sativi include: inoculating a mycelium into a culture solution containing PDB for culture to obtain a seed solution; and inoculating the seed solution into a fermentation medium for static culture at 28° C. for 30 days; where the fermentation medium includes 80 g of oats and 120 ml of seawater with a salinity of 3%.

Further, in the step S1, the mycelium is prepared by the following step: culturing Penicillium allii-sativi on a PDA plate at 28° C. for 3 to 4 days to obtain the mycelium.

According to a third aspect, the present invention provides a use of the benzothiazole meroterpenoid compound, a derivative compound thereof and a derivative thereof or a salt thereof in the preparation of the following products: 1) inhibitors against proliferation of tumor cells; and 2) drugs for preventing and/or treating tumor diseases.

Preferably, the tumor cells include but are not limited to cervical cancer cells, liver cancer cells, breast cancer cells and prostate cancer cells.

Preferably, the tumor diseases include but are not limited to cervical carcinoma, hepatocellular carcinoma, breast carcinoma and prostatic carcinoma.

### Beneficial Effects of the Present Invention

Compared with the conventional technology, the present invention has the following advantages:

The present invention provides three new compounds: meroterpenthiazole A (formula I), 4-(5-hydroxy-7-methylbenzo[d]thiazol-4-yl)-2-methylbutanoic acid (formula II), and 4-(5-hydroxy-4-methylbenzo[d]thiazol-7-yl)-2-methylbutanoic acid (formula III). These compounds are isolated from fermentation broths of deep-sea-derived Penicillium allii-sativi. The compound meroterpenthiazole A is a new skeleton compound composed of sesquiterpenoids and benzothiazole rings, and is a kind of sulfur-containing meroterpenoid secondary metabolites with novel structures. In nature, structures containing thiazole rings are very rare. According to the present invention, a new skeleton compound composed of sesquiterpenoids and benzothiazole rings is discovered for the first time, which is of great significance for discovering and researching new RXRalpha targets. The method for isolating the compounds I to III from fermentation broths has the advantages of environmental protection, simple steps, high product purity, and the like. By means of RXRalpha dual reporter gene experiments, surface plasmon resonance, molecular docking and cytotoxic activity experiments, it has proved that the compounds I to III inhibit the transcriptional activity of an anti-tumor target RXRalpha by means of binding thereto so as to achieve an anti-cancer effect. Therefore, the three new compounds provided in the present invention have good application prospects in the preparation of anti-cancer drugs.

### BRIEF DESCRIPTION OF THE DRAWINGS

Various other advantages and benefits will become clear to those skilled in the art by reading the following detailed description of preferred embodiments. The accompanying drawings are only used for illustrating preferred implementations, and are not construed as a limitation to the present invention.

FIG. 1 shows results of transcriptional activity of compounds I to III of the present invention for RXRalpha.

FIG. 2 shows a result of binding of compound I of the present invention with RXRalpha-LBD.

### DETAILED DESCRIPTION OF THE EMBODIMENTS

### Embodiment 1. Preparation of Benzothiazole Meroterpenoid Compound

(1) Penicillium allii-sativi (collected in Marine Culture Collection of China, with collection No. of MCCC 3A00580) is cultured on a PDA plate at 25° C. for 3 days; then a fresh mycelium is inoculated into a culture solution containing 400 ml of PDB; after 24 h, 10 ml of seed solution is inoculated into a 1 L conical flask (100 flasks), each containing 80 g oat and 120 ml seawater with a salinity of 3%, for static culture at 28° C. for 30 days.
(2) A fermentation product obtained in the step (1) is extracted with ethyl acetate for three times, and an organic solvent is evaporated at a reduced pressure to obtain an organic extract (200 g); after passing through a normal phase chromatographic column, the extract is eluted with petroleum ether, dichloromethane and methanol respectively; and a dichloromethane layer is concentrated to obtain a crude extract (63.0 g).
(3) The crude extract obtained in the step (2) is isolated by normal phase silica gel column chromatography, and gradient elution is performed by using a petroleum ether-ethyl acetate system to obtain eight crude fractions (Fr.1-Fr.8).
(4) The crude fraction Fr.5 (3.5 g) obtained in the step (3) is isolated by ODS column chromatography, and then gradient elution is performed by using a water-methanol system to obtain eleven crude fractions (Fr.5.1-Fr.5.11). The Fr.5.9 (89.1 mg) is isolated by using a sephadex column (pure methanol) and a semi-preparative liquid chromatographic column (acetonitrile-water, 60%→100%) to obtain a compound as represented by formula I (6.2 mg). The Fr.6 (7.0 g) is isolated by using a sephadex column (pure methanol), an ODS column (methanol-water, 40%→100%) and a semi-preparative liquid chromatographic column (acetonitrile-water, 40%→100%) to obtain a compound as represented by formula II (4.8 mg) and a compound as represented by formula III (3.7 mg).
(5) The compounds as represented by formula I, formula II and formula III obtained in the foregoing steps are analyzed by 1D and 2D NMR spectra and high resolution mass spectrometry respectively to determine planar structures of the compounds, and then their absolute configurations are determined by ECD calculation, as detailed below:

The compound as represented by formula I is a white powder whose molecular formula is determined as C₂₆H₃₄N₂O₄S based on its main ion peak in high resolution mass spectrometry. ¹H and ¹¹C NMR data (Table 1), and DEPT and HMBC spectra show 26 carbon signals, including 4 methyl groups, 8 methylene groups, 3 methyne groups and 11 quaternary carbons. A planar structure of the compound is determined based on detailed 2D data. Finally, relative and absolute configurations of the compound as represented by formula I are determined by using NOE spectra, ECD and ¹³C NMR calculation, and the compound is named meroterpenthiazole A.

TABLE 1
¹H and ¹³C NMR data for compounds as represented by formulas I to III

| No | Formula I^{a} | | Formula II ^{b} | | Formula III ^{b} | |
|---|---|---|---|---|---|---|
| | δ_{C} | δ_{H} | δ_{C} | δ_{H} | δ_{C} | δ_{H} |
| 1 | 38.9 t | 1.91 (d, 12.7); 1.22 m | 117.6 s | | 134.4 s | |
| 2 | 19.0 t | 1.59 (dt, 13.6, 2.6) | 152.7 s | | 144.9 s | |
| | | 1.50 (dp, 13.8, 3.5) | | | | |
| 3 | 41.8 t | 1.39 (br d, 13.2) | 116.3 d | 6.85 s | 136.2 s | |
| | | 1.24 (dd, 13.6, 2.6) | | | | |
| 4 | 33.4 s | | 130.8 s | | 113.7 s | |
| 5 | 55.1 d | 1.23 m | 145.5 s | | 152.8 s | |
| 6 | 23.9 t | 1.31 (dq, 12.9, 4.2) | 135.4 s | | 115.1 s | 6.90 s |
| | | 1.72 (br d, 12.8) | | | | |
| 7 | 37.6 t | 2.29 m | 27.7 t | 2.86 m | 29.5 t | 3.07 m |
| | | 1.94 (td, 12.9, 4.8) | — | | — | |
| | | | — | | — | |
| 8 | 147.6 s | | 32.0 t | 1.98 m; 1.72 m | 34.5 t | 2.09 m; 1.83 m |
| 9 | 56.6 d | 2.31 m | 39.2 d | 2.44 m | 39.0 d | 2.48 m |
| 10 | 39.6 s | | 179.3 s | | 179.5* s | |
| 11 | 25.0 t | 3.37 (dd, 15.2, 11.2) | 16.4 q | 1.20 d (7.0) | 16.3 q | 1.19 d (7.0) |
| | | 3.10 (d, 15.1) | | | | |
| 12 | 136.9 s | | 17.0 q | 2.63 s | 13.7 q | 2.38 s |
| 13 | 145.2 s | | 150.6 d | 8.88 s | 150.6 d | 8.94 s |
| 14 | 138.7 s | | | | | |
| 15 | 112.8 s | | | | | |
| 16 | 154.1 s | | | | | |
| 17 | 115.4 d | 6.96 s | | | | |
| 18 | 33.5 q | 0.88 s | | | | |
| 19 | 21.7 q | 0.82 s | | | | |
| 20 | 14.6 q | 0.84 s | | | | |
| 21 | 108.4 t | 4.64 s; 4.66 s | | | | |
| 22 | 14.9 q | 2.27 s | | | | |
| 23 | 157.9 s | | | | | |
| 24 | 160.0 s | | | | | |
| 1′ | 170.9 s | | | | | |
| 2′ | 41.4 t | 4.01 (dd, 17.4, 6.0) | | | | |
| | | 3.98 (dd, 17,4, 5.9) | | | | |
| 2′-NH | | 8.84 (dd, 5.7, 5.3) | | | | |
| 16-OH | | 9.85 br s | | | | |

^{a}DMSO;
^{b} CD₃OD

The compound as represented by formula II has a molecular formula of C₁₃H₁₅NO₃S, and ¹H and ¹³C NMR data (Table 1) show that there are 13 carbons, including 2 methyl groups, 2 methylene groups, 3 methyne groups and 6 quaternary carbons. These signals are similar to some data in the compound as represented by formula I. Through detailed 1D and 2D NMR analysis, the compound as represented by formula H is determined to be 4-(5-hydroxy-7-methylbenzo[d]isothiazol-4-yl)-2-methylbutanoic acid.

The compound as represented by formula III has a molecular formula of C₁₃H₁₅NO₃S. The ¹H and ¹³C NMR data are very similar to those of formula II except for C-4, C-7 and C-12 (Table 1). Through detailed spectral analysis, a structure of the compound is determined to be 4-(5-hydroxy-4-methylbenzo[d]thiazol-7-yl)-2-methylbutanoic acid.

### Embodiment 2. Detection of RXRalpha Dual Reporter Gene Activity: To Detect Whether the Compound Affects the Transcriptional Activity of RXRalpha, and to Preliminarily Investigate Whether the Compound is Likely to Bind to RXRalpha

In this embodiment, a dual-luciferase reporter (DLR) assay system consisting of firefly luciferase (FL) reporter gene and Rellina luciferase (RL) reporter gene is used. The RL reporter gene is used as an internal control to normalize measurement results of the FL reporter gene. In cells lacking endogenous RXRalpha and necessary components for downstream signal transduction, a receptor RXRalpha and reporter gene containing RXRalpha response elements are introduced by transfection, so as to simply simulate a transcriptional activation process of a receptor in vivo.

In this embodiment, the following three groups are set:
Negative control group: culture solution of an equal amount, 0.1% DMSO, containing cells, without the compounds as represented by formulas I to III;
Positive control group: culture solution of an equal amount, added with an RXRalpha agonist 9-cis-retinoic acid with or without an antagonist UVI3003 respectively;
Treatment group: cell culture solutions added with an RXRalpha agonist 9-cis-retinoic acid and the compounds as represented by formulas I to III respectively.

Specific steps include:
(1) Cell culture and preparation of test solution
Human embryonic kidney cells (293T) are cultured in a DMEM medium containing 10% fetal bovine serum (FBS) in an incubator containing 5% CO₂ at 37° C., and passaged regularly based on growth law of the cells. All the cells used in the experiment are in a logarithmic phase. A sample to be tested, 9-cis-retinoic acid (9-cis-RA) and UVI3003 are prepared into a stock solution with DMSO, and the stock solution is diluted to a desired concentration with a culture solution containing 10% FBS before use.
(2) Determination of activity
Human embryonic kidney cells (293T) are inoculated into a 96-well culture plate at 1×10⁴/well and cultured at 37° C. for 24 h; then cells with a 80-90% fusion degree are exchanged, and a target plasmid is transfected into the cells by using a liposome; the cells are dosed (12.5 μM, 25 μM, 50 μM) 24 h after transfection, with an effect lasting for 12 h; then, the cells are washed with PBS, and 40 μL of cell lysis buffer PLB (passive lysis buffer) is added: after shaking on a shaker at an intermediate speed for 20 min, 20 μl of lysis buffer is separated and transferred to a 96-well shading plate; activity of firefly luciferase is measured immediately after 50 μL of luciferase assay reagent H (LARII) is added to each well, and then 50 μL of Stop & Glo™ reagent is added to quench a firefly luciferase reaction and activate a Ranilla luciferase reaction, and activity of Ranilla luciferase is measured immediately.

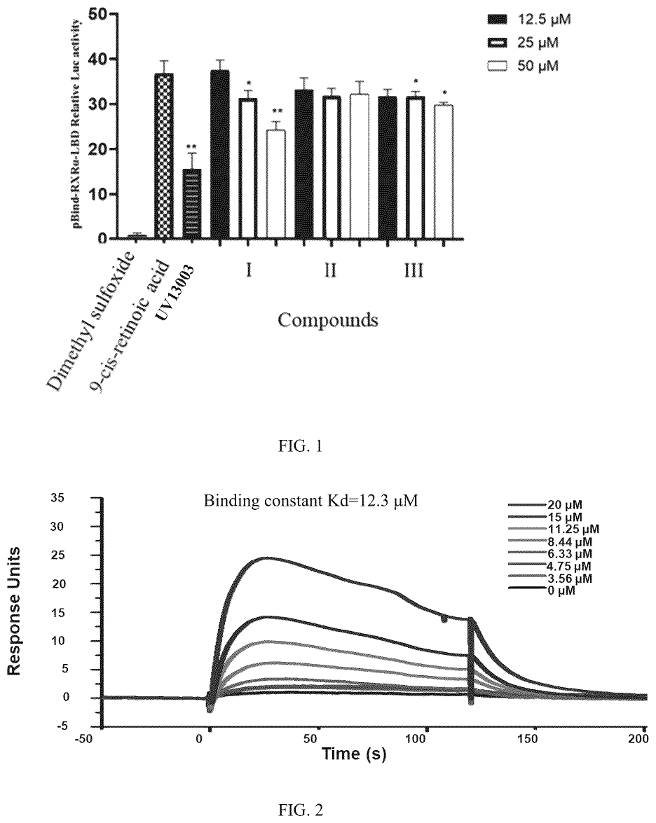

Results are shown in FIG. 1. When used in combination with 9-cis-RA, an agonist of RXRalpha, the compound I can inhibit the transcription of RXRalpha by 9-cis-RA in a concentration-dependent manner.

### Embodiment 3. Biacore Experiment: To Evaluate Whether Compounds I to III Directly Bind to RXRalpha-LBD Protein

A purified nuclear receptor RXRalpha-LBD protein is coupled with a CM5 chip of Biacore. Then the compounds to be tested are diluted with PBS to prepare solutions at different concentrations before sampling. When samples to be tested flow over the surface of the chip, the binding between biomolecules results in an increase in surface mass of a biosensor, so that refractive index changes. By monitoring angular variation of SPR, kinetic binding and dissociation constants, affinity and specificity of analytes can be automatically obtained. A BiacoreT200 detector can detect interaction between a target protein and the samples to be tested in real time, and binding strength is expressed in response unites (RU) (a change in concentration of binding substances on the chip surface by 1 pg/mm2 is defined as 1 RU). The compounds I to III are screened by Biacore technology, and a binding constant K_{D} of small molecular compounds with RXRalpha-LBD is calculated based on a concentration gradient experiment. Results are shown in FIG. 2. The compound I can bind to the RXRalpha protein in a fast binding and fast dissociation mode, with a binding constant of 12.3 μM.

### Embodiment 4. Detection of Cytotoxic Activity of Compounds I to III

In this embodiment, four tumor cell lines are selected: cervical cancer cells (Hela), liver cancer cells (HepG2), breast cancer cells (MDA-MB231), and prostate cancer cells (LNCap). Cytotoxicity of the compounds I to III prepared in Embodiment 1 is detected by detecting inhibition rate of the compound samples on these tumor cells.

In this embodiment, the following three groups are set:
Negative control group: culture solution of an equal amount, 0.1% DMSO, containing cells, without the compounds I to III;
Blank control group: culture solution of an equal amount, cell-free, without the compounds I to III;
Treatment group: cell culture solutions added with the compounds I to III respectively.

Specific steps include:
(1) After routine digestion, cells are resuspended in a culture medium and blown into a single-cell suspension, and then inoculated into a 96-well plate with 2000-5000 cells per well, with a volume of 200 μl per well.
(2) The cells are cultured in an incubator containing 5% CO₂ at 37° C. for 24 h, and then treated with the compounds at different concentrations.
(3) After culture for another 48 h, 10 μl of 5 mg/ml MTT (3-(4,5)-dimethylthiahiazo(-z-yl)-3,5-diphenytetrazoliumromide) is added to each well for reaction in a dark place at 37° C. for 3 h; after the supernatant is carefully pipetted and discarded, 150 μl of DMSO is added to each well and shaken for 10 min to fully dissolve crystals.
(4) An absorbance value at 570 nm is measured by an ELIASA to calculate an inhibition rate.

Conclusion: The compounds I to III have no significant cytotoxicity (IC₅₀>50 μM) to cervical cancer cells (Hela), liver cancer cells (HepG2), breast cancer cells (MDA-MB231), and prostate cancer cells (LNCap).

The foregoing descriptions are merely specific implementations of the present invention, but are not intended to limit the protection scope of the present invention. Any change or replacement readily figured out by a person skilled in the art within the technical scope disclosed in the present invention shall fall within the protection scope of the present invention. Therefore, the protection scope of the present invention shall be subject to the protection scope of the claims.

What is claimed is:

1. A benzothiazole meroterpenoid compound and a derivative of the benzothiazole meroterpenoid compound, wherein the benzothiazole meroterpenoid compound is a compound as represented by formulas (I to III) or a salt of a benzothiazole meroterpenoid compound of formulas (I to III):

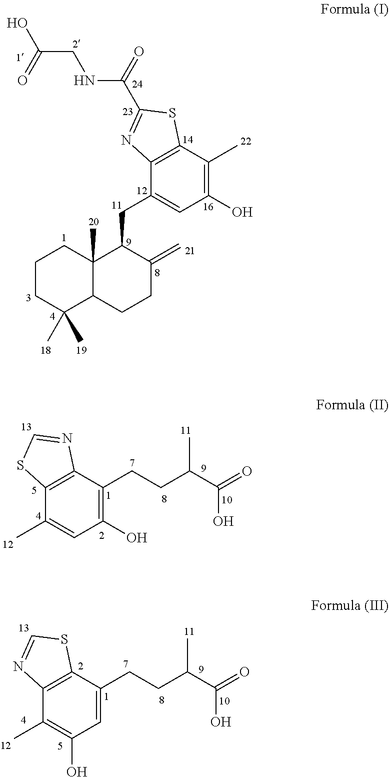

2. A preparation method for the benzothiazole meroterpenoid compound and the derivative of the benzothiazole meroterpenoid compound according to claim 1, comprising the following steps:
S1. Fermenting Penicillium allii-sativi to obtain a fermentation product; wherein the Penicillium allii-sativi is collected in the Marine Culture Collection of China, with a collection No. of MCCC 3A00580; and
S2. Extracting the fermentation product obtained in step S1 with ethyl acetate, and isolating and purifying a resulting extract to obtain the benzothiazole meroterpenoid compound and the derivative of the benzothiazole meroterpenoid compound.

3. The preparation method according to claim 2, wherein the step S2 comprises:
S21. Extracting the fermentation product obtained in the step S1, extracting the fermentation product with ethyl acetate, taking an organic extract for a chromatography, and eluting with petroleum ether, dichloromethane, and methanol, respectively; and concentrating a dichloromethane layer to obtain a crude extract;
S22. Isolating the crude extract obtained in the step S21 by a normal phase silica gel column chromatography to successively obtain eight crude fractions: Fr.1-Fr.8;
S23. Isolating a crude fraction Fr.5 obtained in the step S22 by a first ODS column chromatography to successively obtain eleven crude fractions: Fr.5.1-Fr.5.11;
S24. Isolating a crude fraction Fr.5.9 obtained in the step S23 by using a sephadex column and a fir semi-preparative liquid chromatographic column to obtain a compound as represented by formula I;
S25. Isolating a crude fraction Fr.6 obtained in the step S22 by a sephadex column chromatography to obtain three crude fractions: Fr.6.1-Fr.6.3; and
S26. Isolating the crude fraction Fr.6.2 obtained in the step S25 by using a second ODS column and a second semi-preparative liquid chromatographic column to obtain compounds as represented by formula II and formula III.

4. The preparation method according to claim 2, wherein in the step S1, fermentation conditions for the Penicillium allii-sativi comprise: inoculating a mycelium into a culture solution containing a PDB for a culture to obtain a seed solution; and inoculating the seed solution into a fermentation medium for a static culture at 28° C. for 30 days; wherein the fermentation medium comprises 80 g of oats and 120 ml of seawater with a salinity of 3%.

5. The preparation method according to claim 4, wherein in the step S1, the mycelium is prepared by the following step: culturing the Penicillium allii-sativi on a PDA plate at 28° C. for 3 to 4 days to obtain the mycelium.

6. A method of a use of the benzothiazole meroterpenoid compound and the derivative of the benzothiazole meroterpenoid compound according to claim 1 in a preparation of the following products: 1) inhibitors against tumor cells; and 2) drugs for preventing and/or treating tumor diseases.

7. The method of the use according to claim 6, wherein the tumor cells comprise cervical cancer cells, liver cancer cells, breast cancer cells, and prostate cancer cells.

8. The method of the use according to claim 6, wherein the tumor diseases comprise a cervical carcinoma, a hepatocellular carcinoma, a breast carcinoma, and a prostatic carcinoma.
